(19) United States
(12) Patent Application Publication
(10) Pub. No.: US 2003/0049158 A1
(43) Pub. Date: Mar. 13, 2003

(54) Stablization and terminal sterilization of phospholipid formulations

(76) Inventors: Poh K. Hui, Wellesley, MA (US); Willow R. Diluzio, Westford, MA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 10/102,228

(22) Filed: Mar. 20, 2002

Related U.S. Application Data
(60) Provisional application No. 60/281,057, filed on Apr. 3, 2001.

(51) Int. Cl. A61L 9/00

(52) U.S. Cl. 422/1; 514/75

(57) ABSTRACT

A method for sterilizing a lipid-containing formulation wherein the pH and the ionic strength of the lipid-containing formulation are optionally adjusted and the lipid-containing formulation is subjected to a temperature of between about 126° C. and about 130° C. for a time of between about 2 minutes and about 10 minutes. A stabilizing excipient is optionally added to the lipid-containing formulation.

### FIELD OF THE INVENTION

The present invention relates to methods for the steam sterilization of phospholipid formulations and, in particular, to methods for the sterilization of phospholipid formulations having an optional addition of stabilizing excipients, wherein the phospholipid formulation is subjected to a steam sterilization cycle having a short dwell time at an elevated temperature.

### BACKGROUND OF THE INVENTION

Ultrasound is a diagnostic imaging technique which provides a number of advantages over other diagnostic methodology. Unlike techniques such as nuclear medicine and X-rays, ultrasound does not expose the patient to potentially harmful exposures of ionizing electron radiation that can potentially damage biological materials, such as DNA, RNA, and proteins. In addition, ultrasound technology is a relatively inexpensive modality when compared to such techniques as computed tomography (CT) or magnetic resonance imaging.

The principle of ultrasound is based upon the fact that sound waves will be differentially reflected off of tissues depending upon the makeup and density of the tissue or vasculature being observed. Depending upon the tissue composition, ultrasound waves will either dissipate by absorption, penetrate through the tissue, or reflect back. Reflection, referred to as back scatter or reflectivity, is the basis for developing an ultrasound image. A transducer, which is typically capable of detecting sound waves in the range of 1 MHz to 10 MHz in clinical settings, is used to sensitively detect the returning sound waves. These waves are then integrated into an image that can be quantitated. The quantitated waves are then converted to an image of the tissue being observed.

Despite technical improvements to the ultrasound modality, the images obtained are still subject to further refinement, particularly in regards to imaging of the vasculature and tissues that are perfused with a vascular blood supply. Toward that end, contrast agents are typically used to aid in the visualization of the vasculature and vascular-related organs. In particular, microbubbles or vesicles are desirable as contrast agents for ultrasound because the reflection of sound at an interface created at the surface of a vesicle is extremely efficient. It is known to produce suitable contrast agents comprised of microbubbles by first placing an aqueous suspension (i.e., a bubble coating agent), preferably comprising lipids, into a vial or container. A gas phase is then introduced above the aqueous suspension phase in the remaining portion, or headspace, of the vial. The vial is then shaken prior to use in order to form the microbubbles. It will be appreciated that, prior to shaking, the vial contains an aqueous suspension phase and a gaseous phase. A wide variety of bubble coating agents may be employed in the aqueous suspension phase. Likewise, a wide variety of different gases may be employed in the gaseous phase. In particular, however, perfluorocarbon gases such as perfluoropropane may be used. See, for example, Unger et al., U.S. Pat. No. 5,769,080, the disclosure of which is hereby incorporated in by reference in its entirety.

Phospholipids are ubiquitously present in the human body. For example, 1,2-Dipalmitoyl-sn-Glycero-3-Phosphocholine (DPPC) constitutes a large fraction of human cell membranes, i.e., >50%. Phospholipids are also present in mass on the lung alveolar membranes to prevent air sacks from collapsing. Phospholipids are insoluble in water (or aqueous media in general), and are amphiphilic, i.e., they generally consist of a polar head group that is hydrophilic and two a polar tails that are hydrophobic. In the presence of water, the lipids spontaneously self-assemble to form micelles or liposomes depending on their structures. Since phospholipids are non-toxic and compatible with humans, they are ideal candidates for drug delivery vehicles to carry hard to dissolve therapeutic substances (e.g., peptides, proteins, other macromolecules, and genes) to targets or as a control-release device for sustained release of a drug over a prolonged period of time in vivo through the parenteral route. Furthermore, owing to their amphiphilic properties, phospholipids can also be used as stabilizing agents in preparation of emulsions and microbubble ultrasound contrast agents.

Before phospholipids can be administered to a patient, the phospholipid should be sterilized. However, a phospholipid molecule contains four ester bonds which can undergo hydrolysis in the presence of acid or base. Due to the hydrolytic degradation, most lipid products are aseptically processed, i.e., by sterile filtration, which gives a sterility assurance of one in 10³ for the product. It is highly desirable to have a more rugged process that would give a higher degree of sterility assurance that is equivalent to the terminal sterilization of conventional parenteral products, i.e., the probability of sterility failure is less than one in 10¹² units.

### SUMMARY OF THE INVENTION

The present invention relates to methods for treating lipid-containing formulations. The methods produce lipid-containing formulations having a high degree of sterility assurance. In particular, the methods are capable of providing a degree of sterility assurance that is equivalent or superior to the terminal sterilization of conventional parenteral products, i.e., the probability of sterility failure is less than one in 10¹² units. In addition, the methods of the present invention produce sterile lipid-containing formulations without significantly degrading the lipids which comprise the formulation and without producing significant amounts of impurities.

The methods of the present invention comprise the step of subjecting a lipid-containing formulation to a temperature of between about 126° C. and about 130° C. for a time of between about 2 minutes and about 10 minutes. Preferably, the formulation is subjected to a temperature of about 128° C.±1° C. for a time of about 6±0.5 minutes. In one embodiment, the lipid-containing formulation comprises one or more phospholipids.

A stabilizing excipient is optionally added to the lipid-containing formulation. In one embodiment, the stabilizing excipient comprises a pH buffering agent, such as, for example, sodium phosphate or sodium citrate. Alternatively, or additionally, the stabilizing excipient optionally comprises propylene glycol or glycerin.

The methods of the present invention also optionally comprise the steps of adjusting the pH and/or the ionic strength of the lipid-containing formulation.

Additional features and embodiments of the present invention will become apparent to those skilled in the art in view of the ensuing disclosure and appended claims.

### DETAILED DESCRIPTION OF THE INVENTION

[1] In a first embodiment, the present invention relates to a method for treating a lipid-containing formulation comprising the step of subjecting the formulation to a temperature of between about 126° C. and about 130° C. for a time of between about 2 minutes and about 10 minutes.

[2] In another embodiment, the present invention relates to a method according to embodiment [1] wherein the formulation is subjected to a temperature of about 128±1° C. for a time of about 6±0.5 minutes.

[3] In another embodiment, the present invention relates to a method according to either one of embodiments [1] or [2] comprising the step of introducing the lipid-containing formulation into at least one vial under aseptic conditions.

[4] In another embodiment, the present invention relates to a method according to any one of embodiments [1] to [3] comprising the step of adding a stabilizing excipient to the lipid-containing formulation.

[5] In another embodiment, the present invention relates to a method according to any one of embodiments [1] to [4] wherein the stabilizing excipient comprises a pH buffering agent.

[6] In another embodiment, the present invention relates to a method according to embodiment [5] wherein the pH buffering agent comprises a citrate buffer.

[7] In another embodiment, the present invention relates to a method according to embodiment [5] wherein the pH buffering agent comprises a phosphate buffer.

[8] In another embodiment, the present invention relates to a method according to embodiment [4] wherein the stabilizing excipient comprises propylene glycol.

[9] In another embodiment, the present invention relates to a method according to any one of embodiments [1] to [8] comprising the step of adjusting the pH of the lipid-containing formulation.

[10] In another embodiment, the present invention relates to a method according to any one of embodiments [1] to [9] comprising the step of adjusting the total ionic strength of the lipid-containing formulation.

[11] In another embodiment, the present invention relates to a method according to embodiment [9] wherein the pH of the lipid-containing formulation is adjusted after the ionic strength adjusting step.

The present invention relates to methods for the steam sterilization or autoclaving of pharmaceutical formulations containing phospholipids including, but not limited to, 1,2-Dipalmitoyl-sn-Glycero-3-Phosphocholine (DPPC), 1,2-Dipalmitoyl-sn-Glycero-3-Phosphate Monosodium salt (DPPA), etc., or polymer conjugated phospholipids such as N-(MPEG5000 carbamoyl)-Palmitoyl-sn-Glycero-3-phosphaditeylethanolamine (pegylated DPPE or MPEG5000-DPPE). Terminal sterilization (autoclaving) of parenteral formulations containing phospholipids as surfactants, cosolvents, emulsifiers, or drug delivery vehicles significantly enhances sterility assurance and safety of parenteral products by reducing, for example, the presence of a wide variety of potential microbial contaminants. The combination of a short product dwell time at elevated temperatures (e.g., 2 to 10 minutes at 127° C. to 130° C.) and a stabilizing excipient (e.g., a phosphate or citrate buffer at pH 6.5 and/or propylene glycol) significantly reduces hydrolytic degradation of phospholipids during the sterilization process. Sterilization of the lipid products by the methods of the present invention is capable of achieving a minimum of 12-log reduction of microbial contaminants such as Bacillus stearothermophilus. Stabilizing excipients and terminal sterilization of the product are useful in drug formulations containing lipids, as well as in ultrasound contrast enhancement agents that use phospholipids or liposomes as prodrugs in the generation and stabilization of microbubbles.

The methods of the present invention combine appropriate hydrolysis impeding excipients, such as propylene glycol and glycerin, and/or pH buffering agent, and a sterilization cycle which minimizes the product dwell time (i.e., product exposure time at an elevated autoclaving temperature), such that a 12-log reduction of microbial contaminants can be effectively achieved without adversely affecting the product. Suitable hydrolysis impeding excipients include, for example, 0.1 mL/mL (0.11035 g/mL) propylene glycol, 0.1 mL/mL (0.11262 g/mL) glycerin, 5-25 mM sodium phosphate (pH 6.5), and 5-13 mM sodium citrate (pH 6.5).

Prior to use, the phospholipid formulation is sterilized or autoclaved. The sterilization is performed at a temperature that is sufficiently high and a duration that is sufficiently long to effectuate sterilization without significantly adversely affecting the phospholipid. In one particular embodiment, the sterilization is performed for a time of between about 2 and about 10 minutes at a temperature of between about 127° C. and about 130° C. Preferably, the sterilization is performed for about 6±0.5 minutes at a temperature of about 128±1° C. In one embodiment, the temperature and duration of the sterilization cycle employed is selected to provide a lethality equivalent or in excess of a six log reduction of a biological challenge for aseptically processed phospholipid-containing formulations (i.e., the probability of sterility failure is less than one in 10⁶ units). Preferably, the sterilization cycle is selected to provide a degree of sterility assurance that is equivalent to or higher than the terminal sterilization of conventional parenteral products, i.e., the probability of sterility failure is less than one in 10¹² units.

In another embodiment, the present invention relates to methods for the steam sterilization of pre-shaken ultrasound contrast agents containing liposomes formed from one or more phospholipids. The liposomes may be prepared using any one of a variety of conventional liposome preparatory techniques which will be apparent to those skilled in the art. These techniques include freeze-thaw, as well as techniques such as sonication, chelate dialysis, homogenization, solvent infusion, microemulsification, spontaneous formation, solvent vaporization, French pressure cell technique, controlled detergent dialysis, solvent infusion, solvent injection, and others. The size of the liposomes can be adjusted, if desired, by a variety of procedures including extrusion, filtration, sonication, homogenization, employing a laminar stream of a core of liquid introduced into an immiscible sheath of liquid, and similar methods, in order to modulate resultant liposomal biodistribution and clearance. The foregoing techniques, as well as others, are discussed, for example, in U.S. Pat. No. 4,728,578; U.K. Patent Application GB 2193095 A; U.S. Pat. No. 4,728,575; U.S. Pat. No. 4,737,323; International Application PCT/US85/01161; Mayer et al., Biochimica et Biophysica Acta, Vol. 858, pp. 161-168 (1986); Hope et al., Biochimica et Biophysica Acta, Vol. 812, pp 55-65 (1985); U.S. Pat. No. 4,533,254; Mayhew et al., Methods in Enzymology, Vol. 149, pp. 64-77 (1987); Mayhew et al., Biochimica et Biophysica Acta, Vol 755, pp. 169-74 (1984); Cheng et al, Investigative Radiology, Vol. 22, pp. 47-55 (1987); PCT/US89/05040; U.S. Pat. No. 4,162,282; U.S. Pat. No. 4,310,505; U.S. Pat. No. 4,921,706; and Liposomes Technology, Gregoriadis, G., ed., Vol. I, pp. 29-37, 51-67 and 79-108 (CRC Press Inc, Boca Raton, Fla., 1984). The disclosures of each of the foregoing patents, publications and patent applications are incorporated by reference herein, in their entireties.

Although any of a number of varying techniques can be employed, preferably the liposomes are prepared via a novel method for hydration and dispersion of a lipid blend in an aqueous medium as discussed in published International Application WO 99/36104, which is hereby incorporated by reference in its entirety. Briefly, the process described is a technique for forming small unilamellar vesicles (SUVs). The lipid, or lipid blend, is first dissolved in propylene glycol which is heated to 50-55° C. The lipid blend/propylene glycol mixture is then added to a mixture of sodium chloride, glycerin, and water which was also heated at 50-55° C. This mixture is optionally buffered using sodium phosphate or sodium citrate. The pH is then adjusted to 6-6.5 using sodium hydroxide. For the buffered solutions, sodium chloride is then added to adjust the ionic strength to 0.116. The solutions are then heated to 70-75° C. Larger batches are optionally sterile filtered using, for example, 0.22mm filters.

The materials which may be utilized in preparing the liposomes employed in the methods of the present invention include any of the materials or combinations thereof known to those skilled in the art as suitable for liposome construction. The lipids used may be of either natural or synthetic origin. Such materials include, but are not limited to, lipids such as fatty acids, lysolipids, dipalmitoylphosphatidylcholine, phosphatidylcholine, phosphatidic acid, sphingomyelin, cholesterol, cholesterol hemisuccinate, tocopherol hemisuccinate, phosphatidylethanolamine, phosphatidyl-inositol, lysolipids, sphingomyelin, glycosphingolipids, glucolipids, glycolipids, sulphatides, lipids with ether and ester-linked fatty acids, polymerized lipids, diacetyl phosphate, stearylamine, distearoylphosphatidylcholine, phosphatidylserine, sphingomyelin, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3β-yloxy)-1-thio-β-D-galactopyranoside, digalactosyldiglyceride, 6-(Scholesten-3β-yloxy)hexyl-6-amino-6-deoxy-1-thio-β-D-galactop yranoside, 6-(5-cholesten-3β-yloxy)hexyl-6-amino-6-deoxyl-1-thio-β-D-manno pyranoside, dibehenoyl-phosphatidylcholine, dimyristoylphosphatidylcholine, dilauroylphosphatidylcholine, and dioleoyl-phosphatidylcholine, and/or combinations thereof. Other useful lipids or combinations thereof apparent to those skilled in the art which are in keeping with the spirit of the present invention are also encompassed by the present invention. For example, carbohydrates bearing lipids may be employed for in vivo targeting as described in U.S. Pat. No. 4,310,505. Of particular interest for use in the present invention are lipids which are in the gel state (as compared with the liquid crystalline state) at the temperature at which shaking is performed. The phase transition temperatures of various lipids will be readily apparent to those skilled in the art and are described, for example, in Liposome Technology, Gregoriadis, G., ed., Vol. 1, pp. 1-18 (CRC Press, Inc. Boca Raton, Fla. 1984), the disclosures of which are incorporated herein by reference in their entirety. In addition, it has been found that the incorporation of at least a small amount of negatively charged lipid into any liposome membrane, although not required, is beneficial to providing highly stable liposomes. By at least a small amount, it is meant about 1 mole percent of the total lipid. Suitable negatively charged lipids will be readily apparent to those skilled in the art, and include, for example phosphatidylserine and fatty acids. Most preferred for reasons of the combined ultimate ecogenicity and stability are liposomes prepared from dipalmitoyl-phosphatidylcholine. Each of the foregoing lipids, as well as others which will be readily apparent to those skilled in the art, may be employed in the present sterilization process.

By way of general guidance, dipalmitoyl-phosphatidylcholine liposomes may be prepared by dissolving the lipid in a non-aqueous solvent in which the lipid is soluble, preferably propylene glycol, and then contacting the solution with an aqueous solution to form a liposome suspension.

The liposomes are then optionally placed in a vial, the headspace of the vial is optionally adjusted to contain a predetermined amount of gas, such as, for example, a perfluoropropane gas, and the vial aseptically sealed. For example, the gas is introduced into the headspace within the vial above the liposomes by placing the vial in a lyophilizing chamber, reducing the pressure within the chamber, and then introducing the gas into the chamber.

Prior to use, the phospholipid-containing compounds of the present invention are steam sterilized or autoclaved. The sterilization is performed at a temperature that is sufficiently high and a duration that is sufficiently long to effectuate sterilization without significantly adversely affecting the phospholipid-containing compounds. In one particular embodiment, the sterilization is performed for a time of between about 2 minutes and about 10 minutes at a temperature of between about 126° C. and about 130° C. Preferably, the sterilization is performed for a time of about 6±0.5 minutes at a temperature of about 128±1° C. In one embodiment, the temperature and duration of the sterilization cycle employed is selected to provide a lethality equivalent or in excess of a six log reduction of a biological challenge for aseptically processed phospholipid-containing formulations (i.e., the probability of sterility failure is less than one in 10⁶ units). Preferably, the sterilization cycle is selected to provide a degree of sterility assurance that is equivalent to or higher than the terminal sterilization of conventional parenteral products, i.e., the probability of sterility failure is less than one in 10¹² units.

Once sterilized, the vial can be shaken to form lipid-encapsulated gas microbubbles immediately prior to use.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any subcombination.

Phospholipid-containing formulations were tested to demonstrate the added sterility assurance provided by the methods of the present invention. A blend of lipids were prepared in accordance with the weight percents and concentrations given in Table 1. A 0.375 g aliquot of the lipid blend was then mixed with 51.8 g of propylene glycol. The temperature of the propylene glycol/lipid blend was maintained at 55° C. and periodically swirled until the lipid blend was dispersed into the propylene glycol.

TABLE 1

| Component | Weight Percent (%) | Concentration (mg/mL) |
|---|---|---|
| DPPC | 53.5 | 0.401 |
| MPEG5000-DPPE | 40.5 | 0.304 |
| DPPA | 6.0 | 0.045 |

A phospholipid-containing formulation was then prepared by adding the propylene glycol/lipid blend to an aqueous solution (USP grade) of 6.8 mg/mL of NaCl (USP grade) and 0.1 mL/mL (0.11262 g/mL) of glycerin (USP grade). The final concentrations of components in the phospholipid-containing formulation are given in Table 2. Buffered formulations were prepared by adding sodium phosphate or sodium citrate to the lipid formulation.

TABLE 2

| Component | Concentration |
|---|---|
| NaCl | 4.5-6.8 mg/mL |
| Glycerin | 0.1 mL/mL (0.11262 g/mL) |
| Propylene glycol | 0.1 mL/mL (0.11035 g/mL) |
| Lipid blend | 0.75 mg/mL |
| Sodium Phosphate | 5-25 mM (optional) |
| Sodium Citrate | 5-13 mM (optional) |

The pH of the bulk solution was then adjusted by adding sodium hydroxide or hydrochloric acid to the bulk solution to bring the pH of the solution within a specified pH range of 6.0-7.0. For buffered formulations, the ionic strength of the bulk solution was adjusted by adding sodium chloride to the bulk solution to bring the ionic strength of the bulk solution to 0.116. The pH of the bulk solution was then re-adjusted by adding sodium hydroxide and/or hydrochloric acid to the bulk solution. For unbuffered formulations, 6.8 mg/mL of NaCl was initially added, which is equivalent to an ionic strength of 0.116.

### EXAMPLE 1

The ability of a high temperature, low time process in accordance with the present invention to sterilize lipid formulations was investigated using an unbuffered lipid formulation prepared as described above. Groups of 2 mL vials containing 1.6 mL of the bulk solution were each inoculated with 0.1 mL of Bacillus stearothenmophilus (target nominal population of 10⁶ spores/vial). The inoculated vials were exposed to a series of cycles using either a Finn-Aqua Saturated Steam Autoclave (Model 6912-D or 121224-DP) or a Barriquand Superheated Water Autoclave (Model 1342X). Each vial was aseptically opened and the contents removed with an individually wrapped 1 mL sterile pipet and cultured into an individual 100 mL aliquot of Soybean Casein Digest Broth (SCDB) and then incubated at 55-65° C. for a minimum of seven days observing the cultures for signs of turbidity indicating growth. The results obtained are shown in Table 3 as the number of culture producing vials (# Positive) out of the total number of vials tested (# Tested). The data of Table 3 show that high temperature, low time sterilization cycles are effective in sterilizing the lipid formulation.

TABLE 3

| Temperature (° C.) | Duration (minutes) | # Positive/# Tested |
|---|---|---|
| 128 | 7 | 0/10 ^{a} |
| 127 | 7 | 0/10 ^{a} |
| 127 | 5 | 0/10 ^{a} |
| 127 | 3.5 | 0/10 ^{a} |
| 127 | 5 | 0/72 ^{a,b} |
| 127 | 5 | 0/30 ^{c,d} |

^{a} Sterilization cycle performed using Barriquand Superheated Water Autoclave, Model 1342X.
^{b} Test was performed in six separate runs with 12 vials tested per run.
^{c} Sterilization cycles performed using Finn-Aqua Saturated Steam Autoclave Model 121224-DP.
^{d} Test was performed in three separate runs with 10 vials tested per run.

### EXAMPLE 2

A reverse-phase HPLC method with evaporative light scattering detection was used to examine lipid degradation following sterilization. The lipid formulation was prepared as described in connection with Example 1. The results using high temperature, low time sterilization cycles in accordance with the present invention are given in Tables 4-6. For comparison purposes, results using conventional low temperature, high time cycles are given in Table 7. The concentration of the control (unsterilized) solution are shown in the tables, along with the concentrations of the sterilized solutions. The percent change in concentration (as compared to the concentration of the unsterilized control) of the sterilized solution is calculated and shown. Also shown are the formulation, dwell time and temperature, number of vials sterilized per run, and the calculated theoretical dwell F₀ (a measure of the heat input of the cycle). The data in Tables 4-7 show that the high temperature, low time cycles result in less lipid loss than comparable low temperature, high time cycles (i.e., cycles having similar F₀ values).

TABLE 4

| | | | | DPPE- | | | | | |
|---|---|---|---|---|---|---|---|---|---|
| | Dwell* | # Vials | Theor | PEG5K^{a} | | DPPC^{a} | | DPPA^{a} | |
| Formulation | Time/Temp | Sterilized | Dwell F₀ | mg/mL | % | mg/mL | % | mg/mL | % |
| Lipid Blend | Control | NA | NA | 0.284 | NA | 0.369 | NA | .044 | NA |
| (0.75 mg/mL) | (unsterilized) | | | (±.001) | | (±.013) | | (±.001) | |
| Propylene Glycol | | | | | | | | | |
| (0.1 mL/mL) | | | | | | | | | |
| Glycerin | | | | | | | | | |
| (0.1 mL/mL) | | | | | | | | | |
| NaCl | 130° C./5 min | 100 | 38.8 | 0.280 | −1.4 | 0.346 | −6.2 | .037 | −15.9 |
| (6.8 mg/mL) | | | | (±.002) | | (±.001) | | (±.001) | |
| Water | 130° C./10 min | 100 | 77.6 | 0.273 | −3.9 | 0.338 | −8.4 | .033 | −25.0 |
| | | | | (±.003) | | (±.002) | | (±.006) | |

^{a}mean of 3 determinations
*Sterilization cycles performed using Finn-Aqua Saturated Steam Autoclave Model 6912-D.

TABLE 5

| | | | | DPPE- | | | | | |
|---|---|---|---|---|---|---|---|---|---|
| | Dwell* | # Vials | Theor | PEG5K^{a} | | DPPC^{a} | | DPPA^{a} | |
| Formulation | Time/Temp | Sterilized | Dwell F₀ | mg/mL | % | mg/mL | % | mg/mL | % |
| Lipid Blend | Control | NA | NA | 0.273 | NA | 0.383 | NA | 0.039 | NA |
| (0.75 mg/m) | (unsterilized) | | | (±.002) | | (±.012) | | (±.001) | |
| Propylene Glycol | | | | | | | | | |
| (0.1 mL/mL) | | | | | | | | | |
| Glycerin | 126° C./7 min | 150 | 21.6 | 0.269 | −1.5 | 0.340 | −11.2 | 0.035 | −10.3 |
| (0.1 mL/mL) | | | | (±.005) | | (±.001) | | (±.002) | |
| NaCl | 127° C./7 min | 150 | 27.2 | 0.274 | 0.4 | 0.354 | −7.6 | 0.033 | −15.4 |
| (6.8 mg/mL) | | | | (±.007) | | (±.011) | | (±.001) | |
| Water | 128° C./7 min | 150 | 34.3 | 0.270 | −1.1 | 0.344 | −10.2 | 0.034 | −12.8 |
| | | | | (±.003) | | (±.022) | | (±.001) | |

^{a}mean of 3 determinations
*Sterilization cycles performed using Barriquand superheated water Autoclave Model 1342X.

TABLE 6

| | | | | DPPE- | | | | | |
|---|---|---|---|---|---|---|---|---|---|
| | Dwell* | # Vials | Theor | PEG5K^{a} | | DPPC^{a} | | DPPA^{a} | |
| Formulation | Time/Temp | Sterilized | Dwell F | mg/mL | % | mg/mL | % | mg/mL | % |
| Lipid Blend | Control | NA | NA | 0.296 | NA | 0.380 | NA | 0.044 | NA |
| (0.75 mg/mL) | (unsterilized) | | | (±.004) | | (±.009) | | (±.001) | |
| Propylene Glycol | 127° C./10 min | 75 | 38.9 | 0.292 | −1.4 | 0.331 | −12.9 | 0.039 | −11.4 |
| (0.1 mL/mL) | | | | (±.004) | | (±.019) | | (±.001) | |
| Glycerin | | | | | | | | | |
| (01 mL/mL) | | | | | | | | | |
| NaCl | 128° C./10 min | 75 | 49.0 | 0.291 | −1.7 | 0.341 | −10.3 | 0.039 | −11.4 |
| (6.8 mg/mL) | | | | (±.002) | | (−.013) | | (±.001) | |
| Water | 129° C./10 min | 75 | 61.7 | 0.289 | −2.4 | 0.334 | −12.1 | 0.038 | −13.6 |
| | | | | (±.002) | | (±.016) | | (±.001) | |

^{a}mean of 6 determinations
*Sterilization cycles performed using Barriguard superheated water Autoclave Model 1342X.

TABLE 7

| | | | | DPPE- | | | | | |
|---|---|---|---|---|---|---|---|---|---|
| | Dwell* | # Vials | Theor. | PEG5K^{a} | | DPPC^{a} | | DPPA^{a} | |
| Formulation | Time/Temp | Sterilized | Dwell F₀ | mg/mL | % | mg/mL | % | mg/mL | % |
| Lipid Blend | Control | NA | NA | 0.284 | NA | 0.369 | NA | 0.044 | NA |
| (0.75 mg/mL) | (unsterilized) | | | (±.001) | | (±.013) | | (±.001) | |
| Propylene | 124° C./22 min | 200 | 42.9 | 0.277 | −2.5 | 0.318 | −13.8 | 0.035 | −20.5 |
| Glycol | | | | (±.003) | | (±.013) | | (±.001) | |
| (0.1 mL/mL) | | | | | | | | | |
| Glycerin | 124° C./35 min | 200 | 68.2 | 0.273 | −3.9 | 0.311 | −15.7 | 0.033 | −25.0 |
| (0.1 mL/mL) | | | | (±.002) | | (±.005) | | (±.002) | |
| NaCl | 124° C./35 min | 200 | 68.2 | 0.275 | −3.2 | 0.303 | −17.9 | 0.031 | −29.5 |
| (6.8 mg/mL) | | | | (±.000) | | (±.006) | | (±.001) | |
| Water | 124° C./42 min | 200 | 81.9 | 0.273 | −3.9 | 0.305 | −17.3 | 0.030 | −31.8 |
| | | | | (±.002) | | (±.015) | | (±.000) | |

^{a}mean of 3 determinations
*Sterilization cycles performed using Barriquand superheated water Autoclave Model 1342X.

### EXAMPLE 3

Use of high temperature, low time sterilization cycles in accordance with the present invention in a large scale manufacturing process was also investigated. A 45 L liposomes formulation was prepared by the preferred method as described. The formulation was placed in vials, the headspace of the vials was adjusted to contain perfluoropropane gas, and the vials were aseptically sealed. The vials were then sterilized as indicated in Table 8. The results are shown below in Table 8. The data of Table 8 show that the high temperature, low time cycles can be used in a large-scale process without significant lipid degradation.

TABLE 8

| | Dwell | # Vials | DPPE-PEG5K^{a} | DPPC^{a} | DPPA^{a} |
|---|---|---|---|---|---|
| Formulation | Time/Temp | Sterilized | mg/mL | mg/mL | mg/mL |
| Lipid Blend | unsterilized | NA | 0.28-0.30 | 0.36-0.40 | 0.044-0.047 |
| (0.75 mg/mL) | | | | | |
| Propylene | 128° C./6 min | ˜14,000 | 0.29-0.31 | 0.38-0.39 | 0.040-0.042 |
| Glycol | Unsterilized | NA | 0.31-0.32 | 0.42-0.44 | 0.049-0.051 |
| (0.1 mL/mL) | | | | | |
| Glycerin | 128° C./6 min | ˜14,000 | 0.27-0.31 | 0.36-0.43 | 0.037-0.051 |
| (0.1 mL/mL) | Unsterilized | NA | 0.30-0.31 | 0.42-0.44 | 0.043-0.045 |
| NaCl | 128° C./6 min | ˜14,000 | 0.30-0.30 | 0.39-0.41 | 0.038-0.040 |
| (6.8 mg/mL) | Unsterilized | NA | 0.31-0.33 | 0.41-0.43 | 0.046-0.047 |
| Water | 128° C./6 min | ˜14,000 | 0.32-0.34 | 0.38-0.41 | 0.041-0.044 |

^{a}Range of 6 determinations

### EXAMPLE 4

In addition, the use of buffered lipid formulations was investigated. The buffered formulations were prepared in accordance with the procedure described in connection with Example 1 with the addition of sodium phosphate and sodium citrate buffers. The results are shown in Table 9 below. The data of Table 9 show that formulations containing buffer at pH 6.5 result in less lipid degradation during sterilization as compared to formulations without any buffer.

TABLE 9

| | | | DPPE- | | | | | |
|---|---|---|---|---|---|---|---|---|
| | Dwell* | # Vials | PEG5K^{b} | | DPPC^{b} | | DPPA^{b} | |
| Formulation^{a} | Time/Temp | Sterilized | Mg/mL | % | mg/mL | % | mg/mL | % |
| (unbuffered) | unsterilized | NA | 0.266 | NA | 0.385 | NA | 0.052 | NA |
| 6.8 mg/mL NaCl | | | | | | | | |
| pH 6-6.5, I = 0.116 | 128° C./6 min | ˜100 | 0.258 | −3.0 | 0.335 | −13.0 | 0.045 | −14.0 |
| 5 mM Sodium | unsterilized | NA | 0.272 | NA | 0.396 | NA | 0.064 | NA |
| Citrate | | | | | | | | |
| 5.84 mg/mL NaCl | | | | | | | | |
| pH 6.5, I = 0.116 | 128° C./6 min | ˜100 | 0.262 | −3.6 | 0.369 | −6.9 | 0.061 | −4.7 |
| (unbuffered) | unsterilized | NA | 0.265 | NA | 0.389 | NA | 0.048 | NA |
| 6.8 mg/rnL NaCl | | | | | | | | |
| pH 6-6.5, I = 0.116 | 128° C./6 min | ˜100 | 0.252 | −5.0 | 0.332 | −14.6 | 0.039 | −18.8 |
| 5 mM Sodium | unsterilized | NA | 0.274 | NA | 0.403 | NA | 0.052 | NA |
| Phosphate | | | | | | | | |
| 5.84 mg/mL NaCl | | | | | | | | |
| pH 6.5, I = 0.116 | 128° C./6 min | ˜100 | 0.273 | −0.5 | 0.379 | −5.9 | 0.045 | −14.1 |

^{a}Formulations also contain: 0.75 mg/mL Lipid Blend, 0.1 mL/mL Propylene Glycol, 0.1 mL/mL Glycerin, and water
^{b}mean of 3 determinations
*Sterilization cycles performed using Finn-Aqua Saturated Steam Autoclave Model 6912-D.

### EXAMPLE 5

Lipid degradation of buffered formulations was also studied in a large scale manufacturing process. The results are shown in Table 10 below. The data of Table 10 show that large scale processing of formulations buffered with sodium phosphate or sodium citrate using a high temperature, low time sterilization cycle of the present invention results in only insignificant amounts of lipid degradation.

TABLE 10

| | Dwell* | # Vials | DPPE-PEG5K^{b} | DPPC^{b} | DPPA^{b} |
|---|---|---|---|---|---|
| Formulation^{a} | Time/Temp | Sterilized | mg/mL | mg/mL | mg/mL |
| Theoretical target | N/A | N/A | 0.30 | 0.40 | 0.045 |
| concentration | | | | | |
| (unbuffered) | 128° C./6 min | ˜400 | 0.29-0.30 | 0.37-0.40 | 0.039-0.041 |
| 6.8 mg/mL NaCl | | | | | |
| pH 6-6.5, I = 0.116 | | | | | |
| 25 mM Sodium | 128° C./6 min | ˜400 | 0.28-0.29 | 0.38-0.39 | 0.040-0.041 |
| Phosphate | | | | | |
| 5.18 mg/mL NaCl | | | | | |
| pH 6.5, I = 0.116 | | | | | |
| 13 mM Sodium | 128° C./6 min | ˜400 | 0.28-0.28 | 0.43-0.43 | 0.058-0.060 |
| Citrate | | | | | |
| 4.52 mg/mL NaCl | | | | | |
| pH 6.5, I = 0.116 | | | | | |

^{a}Formulations also contain: 0.75 mg/mL Lipid Blend, 0.1 mL/mL Propylene Glycol, 0.1 mL/mL Glycerin, and water
^{b}range of 3 determinations
*Sterilization cycles performed using Finn-Aqua Saturated Steam Autoclave Model 6912-D.

### EXAMPLE 6

A reverse phase HPLC method with evaporative light scattering detection was also used to determine the presence of known impurities in lipid formulations subjected to high temperature, low time sterilization cycles in accordance with the present invention. The lipid formulations were prepared as described above in connection with Examples I and 4. The impurities detected included palmitic acid, lyso-PC (1-acyl) palmitoyl lysophosphatidyl choline (1-acyl) and mPEG5K-lyso-PE [methoxypolyethylene glycol 5000 palmitoyl lysophosphatidyl ethanolamine(1-acyl)]. The results are shown below in Table 11. Included in the table is the total percent of detected lipid impurities (Total Imp.) which was calculated as: (concentration of impurities/(0.75mg/mL))*100. The data in Table 11 show that only insignificant amounts of impurities were produced by the high temperature, low time cycle for both the buffered and the unbuffered formulations. The data of Table 11 also show that the level of impurities present in the buffered formulations was significantly improved as compared to the unbuffered formulations.

TABLE 11

| | | | | | mPEG5K- | | |
|---|---|---|---|---|---|---|---|
| | | | Palmitic | Lyso-PC | Lyso- | | Total |
| | Dwell* | # Vials | Acid^{b} | (1-acyl)^{b} | PE^{b} | mPEG5K^{b} | Imp.^{b} |
| Formulation^{a} | Time/Temp | Sterilized | (%) | (%) | (%) | (%) | (%) |
| (unbuffered) | Unsterilized | NA | 0.09 | 0.44 | 0.57 | 4.27 | 5.37 |
| 6.8 mg/mL NaCl | control | | | | | | |
| pH 6-6.5, I = 0.116 | | | | | | | |
| | 128° C./6 min | ˜100 | 1.35 | 1.37 | 1.63 | 4.09 | 8.43 |
| 5 mM Sodium Citrate | Unsterilized | NA | 0.05 | 0.36 | 0.37 | 4.81 | 5.59 |
| 5.84 mg/mL NaCl | control | | | | | | |
| pH 6.5, I = 0.116 | | | | | | | |
| | 128° C./6 min | ˜100 | 0.79 | 0.87 | 1.07 | 4.51 | 7.24 |
| (unbuffered) | Unsterilized | NA | 0.17 | 0.61 | 0.00 | 4.44 | 5.21 |
| 6.8 mg/mL NaCl | control | | | | | | |
| pH 6-6.5, I = 0.116 | | | | | | | |
| | 128° C./6 min | ˜100 | 1.56 | 1.49 | 1.47 | 4.32 | 8.83 |
| 5 mM Sodium | Unsterilized | NA | 0.00 | 0.43 | 0.00 | 4.87 | 5.29 |
| Phosphate | control | | | | | | |
| 5.84 mg/mL NaCl | | | | | | | |
| pH 6.5, I = 0.116 | | | | | | | |
| | 128° C./6 min | ˜100 | 0.85 | 0.89 | 0.79 | 4.64 | 7.18 |

^{a}Formulations also contain: 0.75 mg/ML Lipid Blend, 0.1 mL/mL Propylene Glycol, 0.1 mL/mL Glycerin, and water
^{b}mean of 2 values
*Sterilization cycles performed using Finn-Aqua Saturated Steam Autoclave Model 6912-D.

### EXAMPLE 7

The presence of known impurities in lipid formulations subjected to high temperature, low time sterilization cycles in accordance with the present invention was also investigated for a large scale manufacturing process. The results are shown in Table 12 below. The data of Table 12 show that both buffered and unbuffered formulations subjected to a high temperature, low time cycle in accordance with the present invention in a large scale process produced only insignificant levels of impurities.

TABLE 12

| | | | Palmitic | Lyso-PC | mPEG5K- | Total |
|---|---|---|---|---|---|---|
| | Dwell* | # Vials | Acid^{b} | (1-acyl)^{b} | Lyso-PE^{b} | Imp.^{b} |
| Formulation^{a} | Time/Temp | Sterilized | (%) | (%) | (%) | (%) |
| (unbuffered) | 128° C./6 min | ˜400 | 0.84-0.93 | 0.93-0.98 | 0.62-0.69 | 2.4-2.5 |
| 6.8 mg/mL NaCl | | | | | | |
| pH 6-6.5, I = 0.116 | | | | | | |
| 25 mM Sodium Phosphate | 128° C./6 min | ˜400 | 0.76-0.80 | 0.74-0.77 | 0.50-0.50 | 1.5-1.5 |
| 5.18 mg/mL NaCl | | | | | | |
| pH 6.5, I = 0.116 | | | | | | |
| 13 mM Sodium Citrate | 128° C./6 min | ˜400 | 0.77-0.79 | 0.72-0.76 | 0.50-0.54 | 1.4-1.5 |
| 4.52 mg/mL NaCl | | | | | | |
| pH 6.5, I = 0.116 | | | | | | |

^{a}Formulations also contain: 0.75 mg/mL Lipid Blend, 0.1 mL/mL Propylene Glycol, 0.1 mL/mL Glycerin, AND WATER
^{b}range of 3 determinations
*Sterilization cycles performed using Finn-Aqua Saturated Steam Autoclave Model 6912-D.

Those skilled in the art will appreciate that numerous changes and modifications may be made to the preferred embodiments of the invention and that such changes and modifications may be made without departing from the spirit of the invention. It is therefore intended that the appended claims cover all equivalent variations as fall within the true scope and spirit of the invention. For example, there are various other applications for liposomes of the invention, beyond those described in detail herein. Such additional uses, for example, include such applications as hyperthermia potentiators for ultrasound and as drug delivery vehicles. Such additional uses and other related subject matter are described and claimed in PCT patent application WO92/22298 and U.S. Pat. No. 5,209,720, the disclosures of each of which are incorporated herein by reference in their entirety.

What is claimed is:

1. A method for treating a lipid-containing formulation comprising the step of subjecting the formulation to a temperature of between about 126° C. and about 130° C. for a time of between about 2 minutes and about 10 minutes.

2. The method of claim 1 wherein the formulation is subjected to a temperature of about 128±1° C. for a time of about 6±0.5 minutes.

3. The method of claim I comprising the step of introducing the lipid-containing formulation into at least one vial under aseptic conditions.

4. The method of claim 1 comprising the step of adding a stabilizing excipient to the lipid-containing formulation.

5. The method of claim 4 wherein the stabilizing excipient comprises a pH buffering agent.

6. The method of claim 5 wherein the pH buffering agent comprises a citrate buffer.

7. The method of claim 5 wherein the pH buffering agent comprises a phosphate buffer.

8. The method of claim 4 wherein the stabilizing excipient comprises propylene glycol.

9. The method of claim 1 comprising the step of adjusting the pH of the lipid-containing formulation.

10. The method of claim 1 comprising the step of adjusting the total ionic strength of the lipid-containing formulation.

11. The method of claim 10 wherein the pH of the lipid-containing formulation is adjusted after the ionic strength adjusting step.
